(19) United States
(12) Patent Application Publication
(10) Pub. No.: US 2025/0131242 A1
(43) Pub. Date: Apr. 24, 2025

(54) METHODS AND SYSTEMS FOR USE IN TRAIT DEVELOPMENT IN AGRICULTURAL CROPS

(71) Applicant: MONSANTO TECHNOLOGY LLC, Saint Louis, MO (US)

(72) Inventors: Pragya ADHIKARI, Ankeny, IA (US); Jennifer ARP, Saint Louis, MO (US); Alexis-Tzianni CHARALAMPOPOULOS, Boston, MA (US); Megan Eileen GILLESPIE, Wildwood, MO (US); Katie Soyoung HAHM, Irvine, CA (US); Katiana KONTOLATI, Opelika, AL (US); Fabiana Freitas MOREIRA, Saint Louis, MO (US); Silvano Assanga OCHEYA, Farmington, MN (US); Jales Mendes OLIVEIRA FONSECA, Lakeville, MN (US); Ethan Marcus PICKERING, Boston, MA (US); Zhangyue SHI, Stillwater, OK (US); Graziana TARAMINO, Clayton, MO (US)

(21) Appl. No.: 18/915,306

(22) Filed: Oct. 14, 2024

(30) Foreign Application Priority Data
Oct. 19, 2023 (GR) 20230100865

(51) Int. Cl. G16Y 10/05; G06N 3/0442; G06N 3/045; G06N 3/0464; G06N 3/0475; G16B 50/30

(52) U.S. Cl. CPC G06N 3/0442; G06N 3/045; G06N 3/0464; G06N 3/0475; G16B 50/30; G16Y 10/05

(57) ABSTRACT

Example systems and methods are disclosed for use in trait development in agricultural crops. One example computer-implemented method includes identifying multiple proposed varieties of a crop, wherein each of the multiple proposed varieties includes a distinct genetic sequence, as compared to the other ones of the multiple proposed varieties and to known varieties; predict, using a trained model, a trait of interest for each of the multiple proposed varieties based on data included in a repository; select ones of the multiple proposed varieties, based on an acquisition function which is based on phenotypic gain; and cause seeds representative of the selected ones of the multiple proposed varieties to be directed to an experimental phase to assess the trait of interest of the selected ones of the multiple proposed varieties.

### CROSS-REFERENCE TO RELATED APPLICATION

This application claims the benefit of, and priority to, Greek Patent Application No. 20230100865, filed on Oct. 19, 2023. The entire disclosure of the above-referenced application is incorporated herein by reference.

### FIELD

The present disclosure generally relates to methods and systems for use in trait development in agricultural crops.

### BACKGROUND

This section provides background information related to the present disclosure which is not necessarily prior art.

Modification of plants are known to be made, often through selective breeding or genetic manipulation. Based on the particular modifications, resulting plants may exhibit a desired feature (or features). The feature(s) may be tested across a variety of different environments, and when the feature(s) is/are confirmed, the modified plants may be advanced for further development of the plants and/or for commercial implementation, whereby the plants are bulked and sold to growers.

### SUMMARY

This section provides a general summary of the disclosure and is not a comprehensive disclosure of its full scope or all its features.

Example embodiments of the present disclosure generally relate to systems for use in interpreting traits of interest in agricultural crops. In one example embodiment, such a system generally includes a computing device including a memory and at least one processor, wherein the memory includes executable instructions, a trained prediction architecture, and a repository. The repository includes genotypic data for a number of known varieties, and weather and soil data associated with growth of the known varieties. The at least one processor is configured, by the executable instructions and the trained prediction architecture, to: identify multiple proposed varieties of a crop, wherein each of the multiple proposed varieties includes a distinct genetic sequence, as compared to the other ones of the multiple proposed varieties and the known varieties; predict, using the trained model, a trait of interest for each of the multiple proposed varieties based on the data included in the repository; select ones of the multiple proposed varieties, based on an acquisition function which is based on phenotypic gain; and cause seeds representative of the selected ones of the multiple proposed varieties to be directed to an experimental phase to assess the trait of interest of the selected ones of the multiple proposed varieties.

Example embodiments of the present disclosure also generally relate to methods for use in interpreting traits of interest in agricultural crops. In one example embodiment, such a method generally includes identifying multiple proposed varieties of a crop, wherein each of the multiple proposed varieties includes a distinct genetic sequence, as compared to the other ones of the multiple proposed varieties and to known varieties; predict, using a trained model, a trait of interest for each of the multiple proposed varieties based on data included in a repository; select ones of the multiple proposed varieties, based on an acquisition function which is based on phenotypic gain; and cause seeds representative of the selected ones of the multiple proposed varieties to be directed to an experimental phase to assess the trait of interest of the selected ones of the multiple proposed varieties.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments, are not all possible implementations, and are not intended to limit the scope of the present disclosure.

FIG. 1 is an example system of the present disclosure suitable for trait development in agricultural crops;

FIG. 2 is an example architecture that may be used in the system of FIG. 1, where the architecture includes multiple modes of different data in respective mode layers therein for use in interpreting traits in agricultural crops;

FIG. 3 is a block diagram of an example computing device that may be used in the system of FIG. 1; and

FIG. 4 is an example method, suitable for use with the system of FIG. 1, for use in developing and/or creating novel varieties of agricultural crops.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

In connection with agricultural crop development, a number of different techniques may be used to promote desired traits in crops. The techniques often rely on specific decisions, which predict, essentially, the expected performance of the plants being modified, at least to one specific trait (e.g., yield, disease resistance, plant physiology, etc.). In this manner, given hundreds of thousands of potential plants, origins, etc., decisions to account for expected trait performance range into the billions, if not trillions or infinite in number, per experiment. The decisions also become reliant on testing of some of the decisions, which then contribute to other decisions, whereby physical, biological resources allocated to the breeding decisions are often substantial.

Uniquely, the methods and systems herein provide for interpretation of traits of an agricultural crop based on a multi-modal architecture, whereby the substantial number of decisions may be accounted for through the architecture.

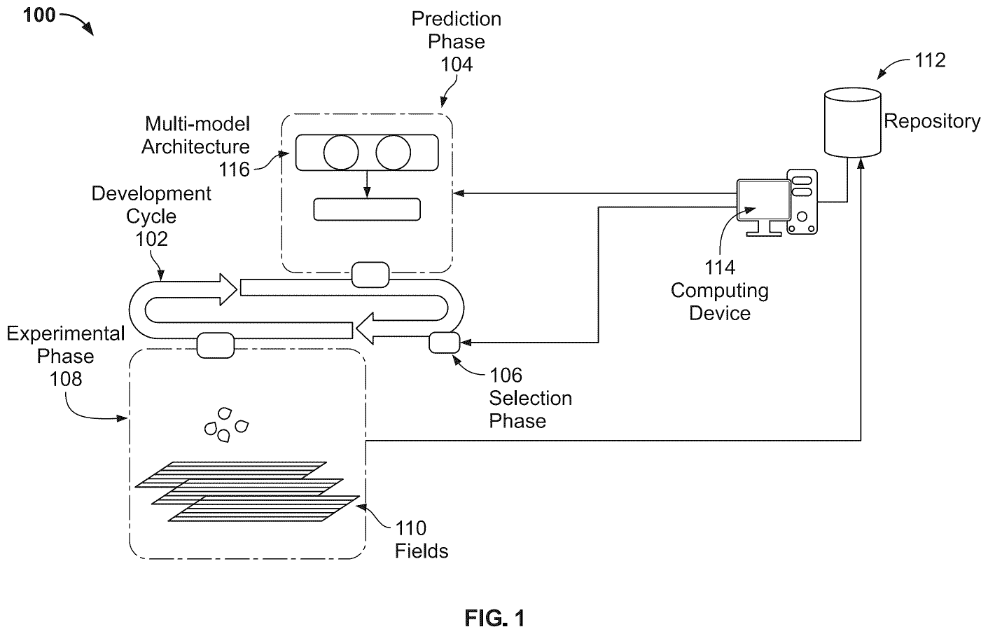

FIG. 1 illustrates an example system 100 for trait integration in agricultural crops based, at least in part, on balancing loss and acquisition features of the agricultural crops. Although, in the described embodiment, parts of the system 100 are presented in one arrangement, other embodiments may include the same or different parts arranged otherwise depending, for example, on available data (e.g., different modes of data, etc.), types of crops, traits of interest, applicable rules and regulations, etc.

As shown in FIG. 1, the system 100 generally includes a crop development cycle 102, which is provided to identify, select, and/or create novel agricultural crops and to select crops to be advanced further experimentation and characterization, etc. (and to ultimately be created, for example). In general, as shown, the crop development cycle 102 includes a prediction phase 104, a selection phase 106 and an experimental phase 108, etc. In this example embodiment, the crop development cycle 102 is configured, by the phases (or stages), to collect data for the performance of crops in fields 110, to store the data in a repository 112, to predict, by a computing device 114, performance of varieties of the crops (e.g., as it relates to at least one phenotypic trait, etc.) (e.g., based on genetic modification thereof, selected environments, etc.) (in the prediction phase 104), and to further select, by the computing device 112, among the novel varieties for the crop development cycle 102 (in the selection phase 106), through physical testing in the fields 110 (in the experimental phase 108).

In general, crops are associated with different genetics, whereby changes or modifications in the genetic sequences of the crops define one variety over another variety. Modifications are made to the genetic sequence of a crop, to define additional varieties, in order to improve the performance of the crop, for example, based on yield, environment, disease resistance, etc. The crop development cycle 102 is configured to be employed in intelligent modification of the genetic sequence to improve the performance of one or more crops.

The crop development cycle 102 may be directed to various different types of crops, which may be any suitable type, etc., of plants. The crops may be of the same type, for example, corn or maize, or may be of different types or varieties, etc. In general, the crops (or plants of the crops) may include, without limitation, soybean (Glycine max), cotton (Gossypium hirsutum), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g., species: Agrostis stolonifera, Poa pratensis, Stenotaphrum secundatum, etc.); wheat (Triticum aestivum), and alfalfa (Medicago sativa), members of the genus Brassica, including broccoli, cabbage, cauliflower, canola, and rapeseed, carrot, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, honeydew melon, cantaloupe and other melons, banana, castorbean, coconut, coffee, cucumber, Poplar, Southern pine, Radiata pine, Douglas Fir, Eucalyptus, apple and other tree species, orange, grapefruit, lemon, lime and other citrus, clover, linseed, olive, palm, Capsicum, Piper, and Pimenta peppers, sugarbeet, sunflower, sweetgum, tea, tobacco, and other fruit, vegetable, tuber, and root crops, etc.

The repository 112 in the system 100 is populated with historical data for a variety of different crops and for different varieties of those crops. Generally, the repository 112 includes genotypic data, weather data, soil data, management data, and phenotypic data.

In connection therewith, the historical data is compiled based, at least in part, of varieties of crops being planted, grown, evaluated and harvested from the fields 110. The fields 110 are suited for growing one or more different types of crops. The fields 110 may include tens, hundreds, thousands or more or less fields, covering tens, hundreds, thousands, or more or less acres (e.g., they may have any suitable size, etc.), individually or in aggregate, etc. The individual ones of the fields 110, accordingly, may be less than an acre in some examples (e.g., which may be referred to as plots, etc.), or more than an acre in other examples, or even more than several acres in still other examples. The fields 110 may be outside and exposed to natural conditions (e.g., weather, etc.), or inside and subject to planned conditions. The fields 110 therefore may include any different types or sizes of growing spaces for the plants (and/or crops).

Additionally, the historical data may include one year or multiple years of data (e.g., Y1, Y2, Y3 . . . . YN, where N is an integer, etc.). In one example, the historical data includes three years, or three growing seasons of data for a specific variety of corn, and five years of data for another variety of corn and only one year of data for yet another variety of corn. The historical data may be organized by variety or crop, type of crop, year, etc.

Specifically, in the repository 112, the genotypic data may include a specific representation of features or markers of one or more sequences of DNA of the varieties of crops. The markers may include any suitable number of base pairs from the sequence(s), and what's more, the sequence(s) may be represented as a specific vector or matrix depending on the specific variety. The genotypic data, then, is an expression of that sequence as a vector of values, where each value represents a marker in the nucleotide sequence. In this example, the vector includes thousands of markers for the male and thousands of markers for the female contributors of the variety, where the location in the vector is indicative of the particular marker and the value in the vector then indicates the content of the marker for that particular sequence of the plant (or crop). Alternatively, genotypic input may include numerical or categorical values that represent genomic features (genomic sequences, genes, genetic alleles, cis-regulatory elements, RNA encoding sequences, etc.) with associated genomic metadata such as gene ontology (GO terms), annotations, expression values, etc. The genotypic data for the variety, generally, is sufficient to reproduce, to some degree, the sequence of the plant 106 represented by the vector.

As indicated above, the varieties of plants (or crops) are planted and grown in the fields 110 (within the experimental phase 108). The varieties are also measured or evaluated (or tested), during growth, at harvest, or otherwise, for one or more types of phenotypic data about the varieties of crops in the fields 110, etc. The phenotypic data may be specific to a trait of the plants/crops, where the trait of interest may include, without limitation, size and/or heartiness (e.g., plant height, car height, standability, sustainability, stalk girth, stalk strength, etc.), yield, time to maturity, resistance to stress(es) (e.g., disease or pest resistance, etc.), resistance to abiotic stress(es) (e.g., drought or salinity resistance, etc.), growing climate, or any other suitable phenotypic data, and/or combinations thereof. The trait of interest may additionally, or alternatively, include, without limitation, yield, thousand kernel weight, saleable seed units, average seed pixel area, tassel size and skeletonization, grey leaf spot, anthracnose, goss's wilt, diplodia, fusarium, gibberella, northern leaf blight, brown/southern rust, tar spot, greensnap, moisture, plant height, lodging, chloride, southern stem canker, white mold, sudden death syndrome, soybean cyst nematode, root knot nematode, phytophthora, iron deficiency chlorosis, frogeye leaf spot, brown steam rot, maturity, and/or combinations thereof.

The phenotypic data is associated with identifying data for the specific variety (e.g., the plant identifiers for the crop, etc.), for example, to identify the specific phenotypic data to the specific variety and/or the specific field 110.

While the varieties are growing in the fields 110, the fields 110 experience various different types of weather, from precipitation to sunshine, to wind, or other conditions, etc. In this example embodiment, the weather data is measured and/or collected to indicate conditions of the fields 110 while, or before, the crops are grown therein. The weather data may be collected from the fields 104 (e.g., via sensors in the fields 110, etc.) or from a third party source. The weather data is stored in the repository 112. The weather data is associated with identifying data for one or more of the fields 110 (e.g., via field identifier, etc.), for example, to identify the specific weather data, over time, to the specific field(s) 110. The weather data, for example, may include, without limitation, atmospheric pressure (e.g., average, minimum, maximum, etc.), wind (e.g., maximum gust, minimum speed, maximum speed, average speed, direction, etc.), precipitation (e.g., rate, maximum rate, average rate, volume, etc.), solar radiation (e.g., total, maximum net radiation, etc.), cloud cover (e.g., average, etc.), snow (e.g., cover, depth, density, etc.), soil temperature at different levels (e.g., levels 1-4, etc.) (e.g., average, minimum, maximum, etc.), soil moisture at different levels, temperature (e.g., minimum, maximum per interval (e.g., day, hour, etc.), etc.), dew point temperature (e.g., average, minimum, maximum, etc.), relative humidity (e.g., average, minimum, maximum, etc.), etc.

The weather data may be expressed in a time-series over a regular or irregular interval, generally from a planting date until observance of the trait of interest (e.g., harvest for yield, etc.).

Similarly, before, during or after the growing of the crops in the fields 110, soil in the fields 110 may experience one or more soil conditions. As above, the soil data is measure and/or collected (e.g., through sensors in the fields 110, etc.), and stored in the repository 112. The soil data is associated with identifying data for the corresponding fields 110 (e.g., via field identifier, etc.), for example, to identify the specific soil data, over time, to the specific field(s). The soil data, for example, may include, without limitation, measurements relating to organic matter (OM), cation exchange capacity (cec), ph (e.g., acidity, etc.), sand content, clay content, silt content, available soil water capacity (volumetric fraction until wilting point, and bulk density, etc., which may be captured at one of more discrete times, or intervals, prior to, during, or after the growing of the plants/crop(s) in the fields 110.

Also, the fields 110 are often subjected to one or more management practices, which are reflected in management data. The management data may include, then, without limitation, irrigation practices, no-till practices, cover crops, treatment applications (e.g., chemical sprays, etc.), planting density, etc. The management data may be representative of the agricultural fields (e.g., the same as or similar to the agricultural fields for the weather data and soil data, or a subset thereof, etc.) over one or multiple years. As above, the management data is collected and stored in the repository 112.

It should be appreciated that the historical data above may be compiled for hundreds or thousands of plants, over multiple growing seasons (e.g., three, five, ten, twenty, or more, etc.). As such, the repository 112 includes a substantial volume of data, which identifies the specific genetics of the varieties to the fields 110 in which they were tested along with the weather data and soil data, and also to the performance of the plants. The repository 112, in this example embodiment, includes tens of millions of data points for hundreds of thousands or millions of plants.

In this example embodiment, as shown in FIG. 1, the prediction phase 104 includes a multi-modal architecture 116. The multi-modal architecture 116 is included in the computing device 114 and is trained to predict at least one phenotypic trait of a variety of crops based on multiple modes of input data. The multi-modal architecture 116 is initially trained based on data included in the repository 112 (e.g., the historical data, etc.).

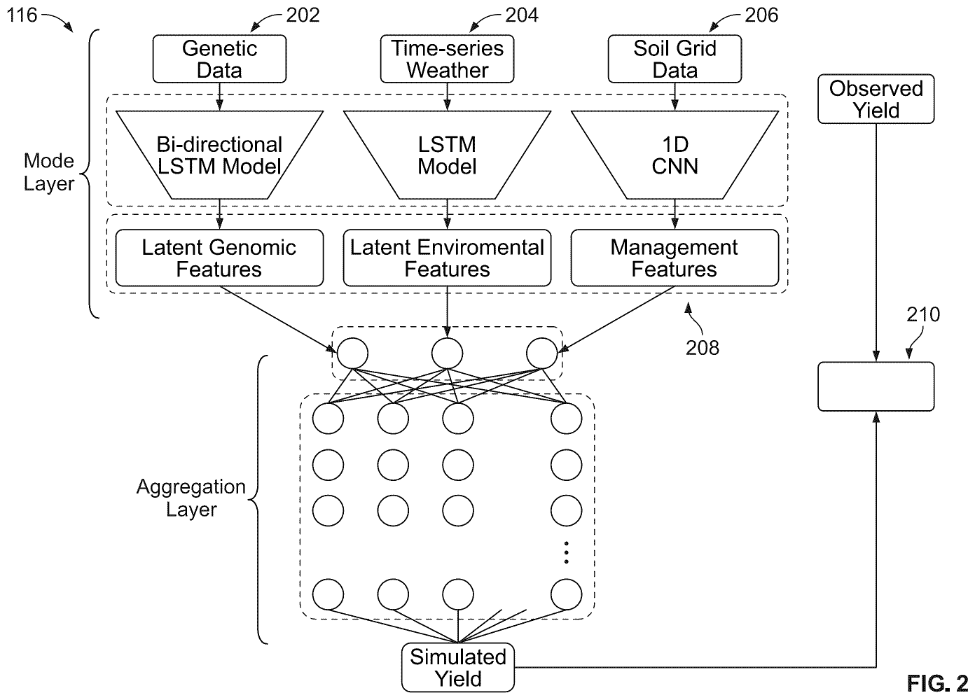

Specifically, the training data includes genotypic data, weather data, soil data, management data, and phenotypic data for historical varieties of crops. Each of the genotypic data, weather data, and soil data defines a mode of the multi-modal architecture 116, and which together define the inputs to the architecture 116. The phenotypic data includes a trait of interest, where a prediction of the trait of interest is the intended output of the architecture 116. The multi-modal architecture 116 is illustrated in more detail in FIG. 2.

As shown, the architecture 116 includes a genetic data mode 202, a weather mode 204, and a soil mode 206 in the mode layer. The genetic mode 202 may include, but is not limited to, a bi-directional long short-term memory (LSTM) model, which is configured to generate latent genetic feature data; the weather mode 204 includes a LSTM model, which is configured to generate latent weather feature data; and the soil mode 206 includes a one-dimensional convolutional neural network (CNN) model, which is configured to generate latent soil feature data. As shown, the latent weather feature data and the latent soil feature data are combined into the latent environmental feature data. In this example, then, the latent genetic feature data, latent environmental feature data, and management practice data 208 are combined in feature fusion between the mode layer and the aggregate layer. The aggregate layer, then, includes a feed-forward neural network with residual connections, which is configured to generate a trait of interest output by combining the latent features from the mode layer.

In other example embodiments, the multi-modal architecture 116 may include other suitable techniques, such as, for example, Bayesian neural networks and conformal predictions, deep neural network (DNN) models, a recurrent neural network (RNN) models, generative pre-trained (GPT) models, multilayer perceptron (MLP) models, etc., or may include one or more different models, per mode, or across multiple modes. U.S. Provisional Application 63/465,239, filed May 9, 2023, includes additional variations of the architecture, which may be employed herein, whereby the entire disclosure of the application is incorporated herein by reference.

It should be appreciated that the multi-modal aspect of the architecture 116 is innovative, important and/or critical, in this example embodiment. By permitting multiple different modes of data to be processed through different models (as described above), in the mode layer, before aggregation in the aggregation layer, the different models of the mode layer are configurable, tailorable and/or selectable to the specific features and/or profile(s) of the data for that specific mode. In this way, in this example embodiment, the disparate data types (e.g., genetic data, weather data, and soil data, etc.) are assessed in the individual models, as explained herein, and combined, whereby the impact of one type of data is not limited or otherwise diminished by the other data (e.g., in that particular models are utilized for the particular different data, etc.), etc.

In connection with the above, as shown in FIG. 2, the architecture 116 also includes a loss-function 210, which is employed to train the architecture 116.

In this example embodiment, the loss function 210 is presented below:

$$L = \frac{1}{\vert D \vert} {\sum }_{G ∈ D} {\frac{{p}_{G} ( G )}{{p}_{y} ( G )} [ {Y}^{ML} ( G ) - Y ( G ) ]}^{2}$$

In the above, D is the entire dataset; G is the genomic representation, or genotype, variable of any arbitrary variety (e.g., a vector of SNPs (single nucleotide polymorphisms)); and Y is the phenotypic observation of interest for the respective variety of G (e.g., yield). And, p_{G}(G)/p_{y}(G) is termed the likelihood weight and represents the importance of high-magnitude and low-probability phenotypes (or events), where p_{y}(G)) is the probability density of the occurrence of a specific phenotype, given G, and is based on labels of training data (e.g., the trait of interest, etc.) and p_{G}(G) is the probability density of the occurrence of a specific genotype, G, given G.

Based on the above, the computing device 114 is configured to train the multi-modal architecture 116, in whole or in parts, based on the training data, so that the multi-modal architecture 116 is predictive of the trait of interest and the loss function is minimized. In this example embodiment, the computing device 114 is configured to input the data from the training data set to each of the modes, i.e., specific data per mode model, to be trained to the known trait of interest from the training data, whereby the architecture 116 is trained in this embodiment as a fully connected architecture. That said, it should be appreciated that in other embodiments, or in connection with updates based on additional data, the different modes of the architecture 116 may be trained individually, and then included in the architecture 116. For example, each of the mode models in the mode layer may be trained separately, based on the specific mode of data in the training data set and the trait of interest, and then, once trained, the mode models may be used, in connection with the aggregation layer, to train the model included in the aggregate layer.

Consistent with the above, the mode layer and the aggregate layer, in combination, may provide for realization of hidden informative features in the intermediate, separate mode data.

Once the multi-modal architecture 116 is trained, the computing device 114 is configured to validate the architecture 116, again, either in parts or as a whole, based on a reserved portion of the training data, i.e., a validation data set. Based on validation that the architecture 116 provides a sufficient performance (e.g., based on one or more thresholds, etc.), the architecture 116 is stored in the repository 112 for use in predicting the trait of interest based on data consistent with the input modes of data. Sufficient performance may be defined based on one or more accuracy levels, deviations, etc. (e.g., compared to a threshold, etc.).

With continued reference to FIG. 1, with the trained (and validated) multi-modal architecture 116, the computing device 114 is configured to identify new genetic sequences for proposed varieties of crops, generally, by modifying the genetic sequence of varieties of crops already included in the repository 112 (e.g., to achieve one or more desired traits of interest, etc.). In connection therewith, in this example, modification of genetic sequences may be performed in two ways: through mating parents or through genome editing. Mating parents leads to large sections of inherited and discarded genetic sequences that the model uses to infer and predict the associated phenotype. As an example, in hybrid crops, such as maize/corn, two inbred parents will lead to a deterministic hybrid offspring that is predicted. Further, an arbitrary number of generations can be assessed in creating novel inbreds that may then be combined for a hybrid representation for evaluation. Genome editing provides a direct change to specific regions that are predicted.

In this manner, the computing device 114 is configured to define hundreds of thousands, or millions or more proposed novel varieties of agricultural crops.

In this example embodiment, the computing device 114 is configured to input the proposed novel varieties into the trained architecture 116, whereby the computing device 114, as configured by the architecture 116, is configured to predict the trait of interest for the proposed novel varieties (e.g., yield, ear height, etc.).

In doing so, the trait of interest is predicted based on the above weather, soil, and/or management data, where the data is indicative of the region of design interest for assessing the novel genetic sequences. For example, a product concept may be defined as a target of the novel proposed varieties. The product concept may include a specific region/location, which may, in turn, define the specific weather data, soil data and also management data (e.g., standard agronomic practices for the region/location, etc.), to be used as an input to the architecture 116 to predict the trait of interest. The product concepts may be further defined and/or may include additional traits of interest or phenotypic performance (e.g., ear height, yield, etc.).

Subsequently, the computing device 114 is configured to select ones of the proposed varieties based on an acquisition function, which may select ones of the proposed varieties which provide for exploration of the genetics thereof and/or exploit certain phenotypic data (e.g., enhanced traits of interest, etc.).

In doing so, in this example, the computing device 114 is configured to evaluate the novel varieties or genomic representations for their potential merit in leading to phenotypic gain (Rₙ), based on:

$${R}_{n} = \frac{Δ P}{{σ}_{P}^{2}}$$

where ΔP is the increase in a maximum observed phenotypic value as compared to an initial maximum observed phenotype before testing future genomic representations and σ_{P}² is the total variance, or uncertainty, of the phenotype integrated over the genomic domain of interest D. Improving phenotypic gain requires special “acquisition”, or field testing, of the identified novel genomic representations. For the express purpose of optimizing, or increasing, phenotypic gain at the quickest rate possible, the acquisition function used for creating and testing the novel genomic representations may be defined as either:

$$α ( G ) = {\overline{y}}_{ENN} ( G ) + κ w ( G ) {σ}_{{y}_{ENN}}^{2} ( G )$$

$$or$$

$$α ( G ) = k {\overline{y}}_{ENN} ( G ) + ( 1 - k ) \frac{{p}_{G} ( G )}{{p}_{{y}_{ENN}} ( G )} {σ}_{{y}_{ENN}} ( G )$$

where y̅_{ENN} (G) is the mean phenotypic value predicted by an ensemble, i.e., multiple realizations, of the models of FIG. 2 for a given genotype G over various environments and management practices (e.g., as defined by a product concept, etc.), κ is a chosen scalar value to weight the importance between increasing y̅_{ENN} (G) and exploring the genetic space D,

$$w ( G ) = \frac{{p}_{G} ( G )}{{p}_{{y}_{ENN}} ( G )}$$

which identifies the importance of high-magnitude and low-probability phenotypes described earlier with the exception that p_{y}_{_{ENN}} is defined by the ensemble of training models, and σ_{y}_{_{ENN}}² (G) is the predictive variance of the phenotype given G and identifies the under-sampled regions of the genetic-phenotypic space. The acquisition function may be defined for one or more different phenotypes, which may then be aggregated for a multi-object acquisition (e.g., as defined through one or more product concepts, etc.). The selected novel genomic representations to create are those which maximize the acquisition value, α. The selected novel genomic representations are then field tested for observations of the phenotype, which, in turn, updates the phenotypic gain equation, both increasing the numerator and decreasing the denominator terms.

In connection with the above, the phenotypic gain may be understood as a metric that is being optimized, or increased, and the acquisition function is then used to achieve such optimization, or increase, in the phenotypic gain.

Next, in the system 100, the selected ones of the proposed varieties are created, as seeds to be planted, and they are then planted in the fields 110, in the experimental phase 108. For instance, seeds and/or plants may be modified via conventional techniques (selective breeding, genetic manipulation, etc.) to produce plants having the sequence of the proposed variety. In connection therewith, as an example, genetic modification of a plant may include point mutations, insertion/deletion mutations, substitution mutations, frameshift mutations, etc. in a plant's genome, which may result in the desired sequence. The “progeny” seeds of a genetically modified “parent” plant, then, may include the genetic modification made in the parent plant and may express the desired phenotypic trait. Conventional sequencing techniques (such as polymerase chain reaction (PCR)) may be used to determine if a progeny seed, or progeny plant produced from said progeny seed, contains the modified sequence. Qualitative observations may also be used to determine if said progeny plant produced from said progeny seed expresses the desired phenotypic trait.

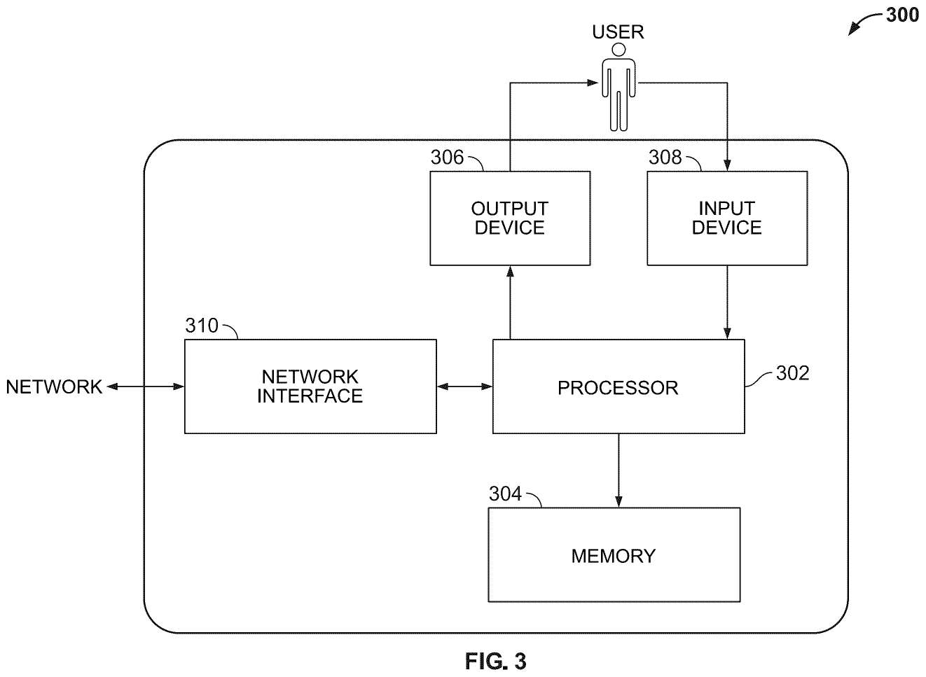

FIG. 3 illustrates an example computing device 300 that may be used in the system 100. In connection therewith, the computing device 114 and/or the repository 112 may include and/or be implemented in at least one computing device consistent with computing device 300. In connection therewith, the computing device 300 may be uniquely, or specifically, configured, by executable instructions, to implement the various algorithms and other operations described herein with regard to the multi-modal architecture 116. It should be appreciated that the system 100, as described herein, may include a variety of different computing devices, either consistent with computing device 300 or different from computing device 300. That said, the repository 112 and the computing device 114 in the system 100 may include and/or may be consistent with the computing device 300.

The example computing device 300 may include, for example, one or more servers, workstations, personal computers, laptops, tablets, smartphones, other suitable computing devices, combinations thereof, etc. In addition, the computing device 300 may include a single computing device, or it may include multiple computing devices located in close proximity or distributed over a geographic region, and coupled to one another via one or more networks. Such networks may include, without limitations, the Internet, an intranet, a private or public local area network (LAN), wide area network (WAN), mobile network, telecommunication networks, combinations thereof, or other suitable network(s), etc. In one example, the repository 112 of the system 100 includes at least one server computing device, which is coupled to the repository 112, directly and/or by one or more LANs, etc.

With that said, the illustrated computing device 300 includes a processor 302 and a memory 304 that is coupled to (and in communication with) the processor 302. The processor 302 may include, without limitation, one or more processing units (e.g., in a multi-core configuration, etc.), including a central processing unit (CPU), a microcontroller, a reduced instruction set computer (RISC) processor, an application specific integrated circuit (ASIC), a programmable logic device (PLD), a gate array, and/or any other circuit or processor capable of the functions described herein. The above listing is example only, and thus is not intended to limit in any way the definition and/or meaning of processor.

The memory 304, as described herein, is one or more devices that enable information, such as executable instructions and/or other data, to be stored and retrieved. The memory 304 may include one or more computer-readable storage media, such as, without limitation, dynamic random access memory (DRAM), static random access memory (SRAM), read only memory (ROM), erasable programmable read only memory (EPROM), solid state devices, flash drives, CD-ROMs, thumb drives, tapes, hard disks, and/or any other type of volatile or nonvolatile physical or tangible computer-readable media. The memory 304 may be configured to store, without limitation, weather data, soil data, genotypic data, latent feature data, phenotypic data, models (e.g., trained, untrained, etc.), and/or other types of data (and/or data structures) suitable for use as described herein, etc. In various embodiments, computer-executable instructions may be stored in the memory 304 for execution by the processor 302 to cause the processor 302 to perform one or more of the functions described herein (e.g., one or more of the operations included in method 400, etc.), such that the memory 304 is a physical, tangible, and non-transitory computer-readable storage media. Such instructions often improve the efficiencies and/or performance of the processor 302 that is performing one or more of the various operations herein. It should be appreciated that the memory 304 may include a variety of different memories, each implemented in one or more of the functions or processes described herein.

In the example embodiment, the computing device 300 also includes an output device 306 that is coupled to (and is in communication with) the processor 302. The output device 306 outputs, or presents, to a user of the computing device 300 (e.g., a breeder, etc.) by, for example, displaying and/or otherwise outputting information such as, but not limited to, selected progeny, progeny as commercial products, traits of interest, performance metrics for plants, and/or any other types of data as desired. It should be further appreciated that, in some embodiments, the output device 306 may comprise a display device such that various interfaces (e.g., applications (network-based or otherwise), etc.) may be displayed at computing device 300, and in particular at the display device, to display such information and data, etc. And in some examples, the computing device 300 may cause the interfaces to be displayed at a display device of another computing device, including, for example, a server hosting a website having multiple webpages, or interacting with a web application employed at the other computing device, etc. Output device 306 may include, without limitation, a liquid crystal display (LCD), a light-emitting diode (LED) display, an organic LED (OLED) display, an “electronic ink” display, combinations thereof, etc. In some embodiments, output device 306 may include multiple units.

The computing device 300 further includes an input device 308 that receives input from the user. The input device 308 is coupled to (and is in communication with) the processor 302 and may include, for example, a keyboard, a pointing device, a mouse, a stylus, a touch sensitive panel (e.g., a touch pad or a touch screen, etc.), another computing device, and/or an audio input device. Further, in some example embodiments, a touch screen, such as that included in a tablet or similar device, may perform as both output device 306 and input device 308. In at least one example embodiment, the output device 306 and the input device 308 may be omitted.

In addition, the illustrated computing device 300 includes a network interface 310 coupled to (and in communication with) the processor 302 (and, in some embodiments, to the memory 304 as well). The network interface 310 may include, without limitation, a wired network adapter, a wireless network adapter, a telecommunications adapter, or other devices capable of communicating to one or more different networks. In at least one embodiment, the network interface 310 is employed to receive inputs to the computing device 300. For example, the network interface 310 may be coupled to (and in communication with) in-field data collection devices (e.g., one or more of the sensors 114, 116, etc.), in order to collect data for use as described herein. In some example embodiments, the computing device 300 may include the processor 302 and one or more network interfaces incorporated into or with the processor 302.

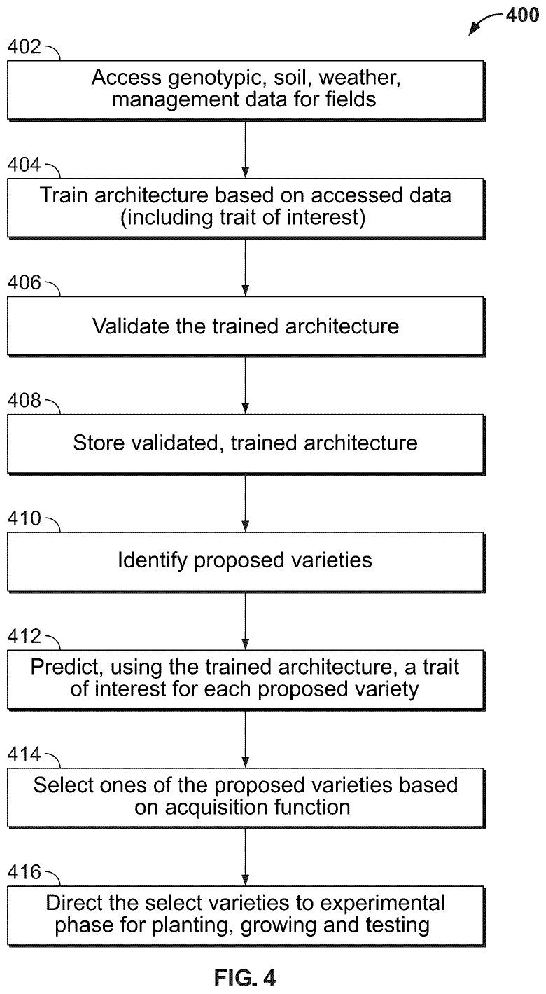

FIG. 4 illustrates an example method 400 for use in facilitating trait development in agricultural crops. The example method 400 is described herein in connection with the system 100, and may be implemented, in whole or in part, in the computing device 114 of the system 100. Further, for purposes of illustration, the example method 400 is also described with reference to the computing device 300 of FIG. 3. However, it should be appreciated that the method 400, or other methods described herein, are not limited to the system 100 or the computing device 300. And, conversely, the systems, data structures/repositories, and computing devices described herein are not limited to the example method 400.

To begin, a plant technician (or other user) (e.g., a breeder, a project manager, etc.) initially identifies a plant type (e.g., maize, soybeans, etc.) and a trait of interest for the plant (e.g., yield, height, etc.). The plant technician may also define a specific environment, for example, depending on the particular aim or variety of the plant (and/or corresponding crop of the plants) to be developed (e.g., in a placement scenario, product concepts, etc.).

In this example, the plant technician has decided to develop one or more varieties of corn, whereby the trait of interest is yield (e.g., where development relates to improving yield of the corn variety, etc.).

Based thereon, at 402, in response to an input by the plant technician (or other user), the computing device 114 accesses data, from the repository 112. The accessed data includes genotypic data, weather data, soil data, management data and trait of interest data for hundreds, thousands, or more varieties of corn over multiple growing seasons. Again, in this example, the trait of interest is yield. Consequently, the accessed data includes, for example, genotypic data, which is indicative of the specific genetic sequence of the variety. The genotypic data may include a numeric vector, in which each value indicates the specific sequence at a particular marker of the genetic sequence. The markers may be limited to those markers known to impact yield and/or additional markers which are known to relate to other traits of interest or unknown as to the impact of one or more traits of interest in the specific crop type.

The weather data, as indicated above, includes a time series of weather data indicative of the specific weather condition(s) of the specific fields (e.g., one or more of fields 110, other fields, etc.) in which the specific varieties resulted in the trait of interest. Likewise, the soil data is indicative of the soil conditions in the fields (e.g., one or more of fields 110, other fields, etc.) in which the variety was grown to provide the trait of interest. In addition to the genotypic data, the weather data and the soil data, management data may be accessed, which is indicative of the management practices, if any, applied to the specific fields (e.g., one or more of fields 110, other fields, etc.) giving rise to the trait of interest.

It should be appreciated that the genotypic data, the weather data, and/or the soil data may be processed to permit and/or to enhance compatibility with the multi-modal architecture 116. In connection therewith, the data may be filtered to only include certain genotypic data, or certain weather data (e.g., average temperature, etc.), and/or certain soil data, etc.

At 404, the computing device 114 trains the architecture 116. In connection therewith, in this example, each of the models in the mode layer is trained separately (e.g., trained separately and then combined via feature fusion, trained together, etc.). The model for the genetic mode 202, or the bi-directional LSTM model in FIG. 2, is trained based on the genetic data and the trait of interest. The model for the genetic mode 202, accordingly, is trained to generate latent feature genotypic data, from the genetic data, whereby the latent feature genotypic data is associated with a specific yield (apart from weather or soil). Likewise, the LSTM model of the weather mode 204 is trained based on the weather data and the trait of interest, to generate latent feature weather data, from the weather data, whereby the latent feature weather data is associated with a specific yield. And, further, the one-dimensional CNN model of the soil mode 206 is trained based on the soil data and the trait of interest, to generate latent feature soil data, from the soil data, whereby the latent feature soil data is associated with a specific yield (apart from genetics or weather).

It should be appreciated that, as part of the training, each trained model from the mode layer may be validated to ensure sufficient performance, as defined, for example, by a percentage, deviation, etc. (e.g., relative to a threshold, etc.).

Further, as part of the training in method 400 in FIG. 4, the trained models, for example, are included in the architecture 116, through which latent feature data of weather and soil modes are combined, and then the training data set is provided, by the computing device 114, to the architecture 116 to train the neural network model of the aggregate layer in combination with the trained models of the mode layer. The overall training of the architecture 116 relies on the loss function described above to modify the hyperparameters and/or weighting of the models included in the architecture 116.

After the neural network model is trained, the computing device 114 validates the overall architecture 116 based on a reserved portion of the training data, at 406. At 408, the trained architecture 116 is stored in memory, such as for example, the repository 112, by the computing device 114. In this manner, the architecture 116 is trained to predict the trait of interest, which is yield, in this example, based on an input of genotypic data, weather data and soil data.

Next, at 410, the computing device 114 identifies genetic sequences for proposed varieties of crops, generally, by modifying the genetic sequence of varieties of crops already included in the repository 112. The computing device 114 inputs the proposed varieties, and in particular, the genotypic data associated therewith along with suitable weather and soil data (and management data) into the trained architecture 116, whereby the computing device 114 uses the trained architecture to predict, at 412, the trait of interest for the proposed varieties.

At 414, the computing device 114 selects ones of the proposed varieties based on, among other things, the above described acquisition function.

Next, at 416, the computing device 114 directs the ones of the proposed varieties to the experimental phase 108, whereby the ones of the proposed varieties are created as physical plants, grown in the fields 110, and tested to verify and/or assess the performance of the trait of interest.

The method 400 may be repeated, with or without retraining, from time to time to continue to explore new varieties of the crop.

In view of the above, the unique systems and methods described herein provide for advanced interpretation of traits of interest, which may be used to define specific genetic profiles, or environmental profiles, for plants.

In particular, in the embodiments herein, technology underlying the interpretation of traits of interest is improved, through inclusion of a multi-modal mode layer and an aggregation layer to permit specific processing, through the mode layer models, of widely disparate types of data (e.g., marker-based genetic data, time-based weather data, and depth-based soil data, etc.). In this manner, the disparate data is accurately and precisely relied on in predicting specific traits of interest (e.g. corn car height, etc.), yet without forfeiting or diminishing impact of any one of the one or more data types to the other one or more data types, as with conventional single model, linear model, etc., approaches, where the data is combined before being introduced or input to the model. This serves to improve the underlying technology of the architecture herein and the resulting predictions therefrom.

With that said, it should be appreciated that the functions described herein, in some embodiments, may be described in computer executable instructions stored on a computer readable media, and executable by one or more processors. The computer readable media is a non-transitory computer readable media. By way of example, and not limitation, such computer readable media can include RAM, ROM, EEPROM, CD-ROM or other optical disk storage, magnetic disk storage or other magnetic storage device, or any other medium that can be used to carry or store desired program code in the form of instructions or data structures and that can be accessed by a computer. Combinations of the above should also be included within the scope of computer-readable media.

It should also be appreciated that one or more aspects of the present disclosure transform a general-purpose computing device into a special-purpose computing device when configured to perform the functions, methods, and/or processes described herein.

As will be further appreciated based on the foregoing specification, the above-described embodiments of the disclosure may be implemented using computer programming or engineering techniques, including computer software, firmware, hardware or any combination or subset thereof, wherein the technical effect may be achieved by performing at least one of the operations recited in the claims, for example: (a) identifying multiple proposed varieties of a crop, wherein each of the multiple proposed varieties includes a distinct genetic sequence, as compared to the other ones of the multiple proposed varieties and the known varieties; (b) predicting, using the trained model, a trait of interest for each of the multiple proposed varieties based on the data included in the repository; (c) selecting ones of the multiple proposed varieties, based on an acquisition function which is based on phenotypic gain; and/or (d) causing seeds representative of the selected ones of the multiple proposed varieties to be directed to an experimental phase to assess the trait of interest of the selected ones of the multiple proposed varieties.

Examples and embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail. In addition, advantages and improvements that may be achieved with one or more example embodiments disclosed herein may provide all or none of the above mentioned advantages and improvements and still fall within the scope of the present disclosure.

Specific values disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may also be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When a feature is referred to as being “on,” “engaged to,” “connected to,” “coupled to,” “associated with,” “in communication with,” or “included with” another element or layer, it may be directly on, engaged, connected or coupled to, or associated or in communication or included with the other feature, or intervening features may be present. As used herein, the term “and/or” and “at least one of” includes any and all combinations of one or more of the associated listed items.

None of the elements recited in the claims are intended to be a means-plus-function element within the meaning of 35 U.S.C. § 112 (f) unless an element is expressly recited using the phrase “means for,” or in the case of a method claim using the phrases “operation for” or “step for.”

Although the terms first, second, third, etc. may be used herein to describe various features, these features should not be limited by these terms. These terms may be only used to distinguish one feature from another. Terms such as “first,” “second,” and other numerical terms when used herein do not imply a sequence or order unless clearly indicated by the context. Thus, a first feature discussed herein could be termed a second feature without departing from the teachings of the example embodiments.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system for use in interpreting traits of interest in agricultural crops, the system comprising:
a computing device including a memory and at least one processor;
wherein the memory includes executable instruction, a trained prediction architecture, and a repository, the repository including genotypic data for a number of known varieties, and weather and soil data associated with growth of the known varieties;
wherein the at least one processor is configured, by the executable instructions and the trained prediction architecture, to:
identify multiple proposed varieties of a crop, wherein each of the multiple proposed varieties includes a distinct genetic sequence, as compared to the other ones of the multiple proposed varieties and the known varieties;
predict, using the trained model, a trait of interest for each of the multiple proposed varieties based on the data included in the repository;
select ones of the multiple proposed varieties, based on an acquisition function which is based on phenotypic gain; and
cause seeds representative of the selected ones of the multiple proposed varieties to be directed to an experimental phase to assess the trait of interest of the selected ones of the multiple proposed varieties.

2. The system of claim 1, wherein the at least one processor is configured, by the executable instructions and the trained prediction architecture, in order to predict the trait of interest, to input genotypic data representative of the proposed varieties along with weather data and soil data associated with a specific region for which the proposed varieties are designated.

3. The system of claim 1, further comprising multiple fields of the experimental phase, wherein the seeds are planted on the multiple fields.

4. The system of claim 1, wherein the at least one processor is configured, by the executable instructions, to train the prediction architecture based on a loss function indicative of phenotypic observations and associated likelihood of high-magnitude and low-probability phenotypic values.

5. The system of claim 1, wherein the prediction architecture includes a multi-modal architecture, which includes a first mode specific to genotypic data, a second mode specific to weather data and a third mode specific to soil data.

6. The system of claim 5, wherein each of the modes includes at least one of: a deep neural network (DNN) model, a convolutional neural network (CNN) model, a recurrent neural network (RNN) model, a generative pre-trained (GPT) model, transformer or language model, a multilayer perceptron (MLP) model, and a long short-term memory (LSTM) model.

7. The system of claim 5, wherein the multi-modal architecture further includes an aggregation layer, which is configured to combine latent features from each of the modes.

8. The system of claim 1, wherein the aggregation layer includes a neural network model.

9. A computer-implemented method for use in interpreting traits of interest in agricultural crops, the method comprising:
identifying, by a computing device, multiple proposed varieties of a crop, wherein each of the multiple proposed varieties includes a distinct genetic sequence, as compared to other ones of the multiple proposed varieties of the crop and as compared to known varieties of the crop;
predicting, by the computing device using a trained prediction architecture, a trait of interest for each of the multiple proposed varieties;
selecting ones of the multiple proposed varieties, based on an acquisition function which is based on phenotypic gain; and
causing seeds representative of the selected ones of the multiple proposed varieties to be directed to an experimental phase to assess the trait of interest of the selected ones of the multiple proposed varieties.

10. The computer-implemented method of claim 9, wherein predicting the trait of interest includes predicting the trait of interest based on genotypic data representative of the proposed varieties along with weather data and soil data associated with a specific region for which the proposed varieties are designated.

11. The computer-implemented method of claim 9, further comprising planting the seeds representative of the selected ones of the multiple proposed varieties in multiple fields of the experimental phase.

12. The computer-implemented method of claim 9, further comprising training the prediction architecture based on a loss function indicative of phenotypic observations and associated likelihood of high-magnitude and low-probability phenotypic values.

13. The computer-implemented method of claim 9, wherein the prediction architecture includes a multi-modal architecture, which includes a first mode specific to genotypic data, a second mode specific to weather data and a third mode specific to soil data.

14. The computer-implemented method of claim 13, wherein each of the modes includes at least one of: a deep neural network (DNN) model, a convolutional neural network (CNN) model, a recurrent neural network (RNN) model, a generative pre-trained (GPT) model, transformer or language model, a multilayer perceptron (MLP) model, and a long short-term memory (LSTM) model.

15. The computer-implemented method of claim 13, wherein the multi-modal architecture further includes an aggregation layer; and wherein the method further comprises combining, by the aggregation layer, latent features from each of the modes.

16. A non-transitory computer-readable storage medium including executable instructions, which, when executed by at least one processor to interpret traits of interest in agricultural crops, cause the at least one processor to:
identify multiple proposed varieties of a crop, wherein each of the multiple proposed varieties includes a distinct genetic sequence, as compared to other ones of the multiple proposed varieties of the crop and as compared to known varieties of the crop;
predict, using a trained prediction architecture, a trait of interest for each of the multiple proposed varieties;
select ones of the multiple proposed varieties, based on an acquisition function which is based on phenotypic gain; and
cause seeds representative of the selected ones of the multiple proposed varieties to be directed to an experimental phase to assess the trait of interest of the selected ones of the multiple proposed varieties.

17. The non-transitory computer-readable storage medium of claim 16, wherein the executable instructions, when executed by the at least one processor, cause the at least one processor to predict the trait of interest, using the trained prediction architecture, based on genotypic data representative of the proposed varieties along with weather data and soil data associated with a specific region for which the proposed varieties are designated.

18. The non-transitory computer-readable storage medium of claim 16, wherein the executable instructions, when executed by the at least one processor, further cause the at least one processor to train the prediction architecture based on a loss function indicative of phenotypic observations and associated likelihood of high-magnitude and low-probability phenotypic values.

19. The non-transitory computer-readable storage medium of claim 16, wherein the prediction architecture includes a multi-modal architecture, which includes a first mode specific to genotypic data, a second mode specific to weather data and a third mode specific to soil data; and
wherein each of the modes includes at least one of: a deep neural network (DNN) model, a convolutional neural network (CNN) model, a recurrent neural network (RNN) model, a generative pre-trained (GPT) model, transformer or language model, a multilayer perceptron (MLP) model, and a long short-term memory (LSTM) model.

20. The non-transitory computer-readable storage medium of claim 19, wherein the multi-modal architecture further includes an aggregation layer; and wherein the executable instructions, when executed by the at least one processor, using the aggregation layer, cause the at least one processor to combine latent features from each of the modes.
